(19) United States
(12) Patent Application Publication
(10) Pub. No.: US 2009/0170854 A1
(43) Pub. Date: Jul. 2, 2009

(54) New Use

(76) Inventor: Arne Eek, Sodertalje (SE)

Correspondence Address: WHITE & CASE LLP;PATENT DEPARTMENT, 1155 AVENUE OF THE AMERICAS, NEW YORK, NY, 10036, US

(21) Appl. No.: 12/185,514

(22) Filed: Aug. 4, 2008

Related U.S. Application Data
(63) Continuation of application No. 10/469,906, filed on Sep. 5, 2003, now abandoned.

(30) Foreign Application Priority Data
Mar. 8, 2001 (SE) 0100798-8
Oct. 3, 2001 (SE) 0103291-1

(51) Int. Cl. A61K 31/501; C07D 471/04; A61K 31/437; C07D 401/04; A61K 31/506; C07D 471/14; A61K 31/4375; C07D 487/04; A61P 1/04

(52) U.S. Cl. 514/248; 546/121; 514/300; 544/323; 514/275; 546/82; 514/293; 544/236

(57) ABSTRACT

The present invention relates to a new use of certain pharmaceutically active compounds in the treatment and/or prevention of medicament induced gastric ulcer. More particularly the invention is directed to the use of said compounds, and pharmaceutically acceptable salts thereof, for the treatment and/or prevention of NSAID (non-steroidal antiinflammatory drugs) induced gastric ulcer as well as a pharmaceutical composition in the unit dosage form for the prevention of NSAID induced gastric ulcer in a mammal comprising an NSAID together with a 6-carboxamido-imidazo[1,2-a]pyridine compounds. Other pharmaceutically active compounds used in the present invention comprises COX-2 inhibitors, NO-NSAIDs and bisphosphonates.

### FIELD OF THE INVENTION

The present invention relates to a new use of certain pharmaceutically active compounds in the treatment and/or prevention of medicament induced gastric ulcer. More particularly the invention is directed to the use of said compounds, and pharmaceutically acceptable salts thereof, for the treatment and/or prevention of NSAID (non-steroidal antiinflammatory drugs) induced gastric ulcer as well as a pharmaceutical composition in the unit dosage form for the prevention of NSAID induced gastric ulcer in a mammal comprising an NSAID together with a 6-carboxamido-imidazo[1,2-a]pyridine compounds.

### BACKGROUND OF THE INVENTION AND PRIOR ART

Certain pharmacological agents are known to be useful in exerting a cytoprotective effect on the gastrointestinal tract. This cytoprotective effect is manifest in the ability of such compounds to treat or prevent inflammatory diseases of the gastrointestinal tract, such as gastric ulcer, duodenal ulcer, gastritis, and intestinal inflammatory diseases, such as Crohn's disease and inflammatory bowel disease.

These inflammatory diseases are known to be caused by a wide variety of agents present in the gastrointestinal tract which are known to attack the surfaces thereof, producing the inflammatory disease response. Such agents include microorganisms, bacterial toxins, certain pharmaceuticals and chemical agents and indeed gastric acid itself is capable of attacking the stomach lining and producing the inflammatory state.

NSAID are a class of compounds that are used to relieve some symptoms caused by arthritis, such as inflammation, swelling, stiffness, and joint-pain. NSAIDs are also used to relieve other kinds of pain or to treat other painful conditions, such as gout attacks, bursitis, tendinitis, sprains, strains, or other injuries.

Any NSAID is known to cause side effects, especially when it is used for a long time or in large doses. One example of such side effects is induced gastric ulcer. COX-2 inhibitors, the newest class of NSAIDS, work by blocking COX-2 enzyme which is involved in the inflammation pathway. By sparing COX-1 enzyme, gastrointestinal toxicity is reduced, but still present.

Nitric oxide (NO) is a molecule of versatility and importance in many guises. In the atmosphere it is a noxious chemical, but in the body in small and controlled doses it is extraordinary beneficial. It helps maintain blood pressure by dilating blood vessels, helps kill foreign invaders in the immune response, is a major biochemical mediator of penile erections, and is proposed to be a major biochemical component of long-term memory. Nitric oxide releasing NSAIDs (NO-NSAIDs) are disclosed in e.g. WO 94/04484.

Bishosphonates are a class of compounds well known for their therapeutic benefits in a variety of disorders associated with abnormal bone resorption, e.g. osteoporosis, Paget's disease, periprosthetic bone loss or osteolysis, metastatic bone disease, hypercalcemia of malignancy, multiple myeloma, periodontal disease and tooth loss. The most common of these disorders is osteoporosis, which in its most frequent manifestation occurs in postmenopausal woman. Examples of such bisphosphonate compounds is alendronate, risedronate, tiludronate, ibandronate, zoledronate and etidronate. Despite their therapeutic benefits, bisphosphonates are poorly absorbed from the gastrointestinal tract. If oral administration of the bisphosphonate is desired relatively high doses must be administered to compensate for the low bioavailability from the gastrointestinal tract. However oral administration of high doses of bisphosphonates are associated with adverse gastrointestinal effects, especially those relating to the esophagus. Pamidronate has for example been associated with esophageal ulcers, see E. G. Lufkin et al., Pamidronate: An Unrecognized Problem in Gastrointestinal Tolerability, Osteoporosis International, 4: 320-322 (1994).

For the treatment of ulcer disease, various drugs such as antacid, anticholinergic agent, Hz-receptor antagonist and proton pump inhibitor have been used. The commercial success of omeprazole has rekindled the interest in this field. The proton pump inhibition by omeprazole is irreversible and a reversible proton pump inhibitor has been suggested to have therapeutical benefits and thus attempts to develop a reversible proton pump inhibitor have been made. For example WO 96/05177 disclose certain 1,2,3,4-tetra-hydroisoquinolin-2-yl)pyrimidine compounds as a reversible proton pump inhibitor.

Further, tricyclic imidazo[1,2-a]pyridine compounds in WO 94/14795 have also been reported and pyrrolopyridazine compounds in EP 742 218.

Certain 6-carboxamido-imidazo[1,2-a]pyridine compounds, as well as methods for producing said compounds, is described in WO 99/55706 and WO99/55705. Said compounds, and pharmaceutically acceptable salts thereof, is said to be effective in inhibiting secretion of gastric acid.

It has now surprisingly been found that certain pharmaceutically active compounds are useful in treatment and/or prevention of gastric ulcer induced by medicaments such as NSAID, COX-2 inhibitors, NO-NSAID and bisphosphonates.

### DESCRIPTION OF THE INVENTION

The present invention relates to the use of certain pharmaceutically active compounds in the treatment and/or prevention of medicament induced gastric ulcer. The present invention can thus be used to prevent a common side-effect affecting users of these pharmaceutically effective compounds. This is easiest done by co-administration of the two medicaments.

One object of the present invention is thus the use of certain 6-carboxamido-imidazo[1,2-a]pyridine compounds, as well as pharmaceutically acceptable salts thereof, of the general Formula I

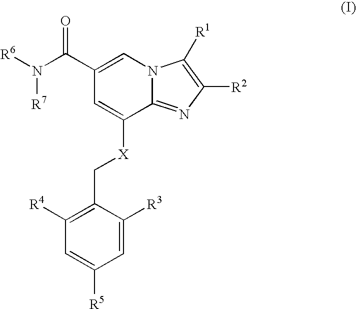

wherein R¹ is
(a) H,
(b) CH₃, or
(c) CH₂OH;

### R² is

(a) CH₃, or
(b) CH₂CH₃;

### R³ is

(a) H,
(b) C₁-C₆ alkyl,
(c) hydroxylated C₁-C₆ alkyl, or
(d) halogen;

### R⁴ is

(a) H,
(b) C₁-C₆ alkyl,
(c) hydroxylated C₁-C₆ alkyl, or
(d) halogen;

### R⁵ is

(a) H, or
(b) halogen;

R⁶ and R⁷ are independently selected substituents, containing C, H, N, O, S, Se, P and halogen atoms, which give compounds of Formula I a molecular weight≦600,

### X is

(a) NH, or
(b) O,
   in the prevention of medicament induced gastric ulcer.

In a preferred embodiment of the present invention, R¹ is CH₃ or CH₂OH; R² is CH₃, R³ is CH₃ or CH₂CH₃; R⁴ is CH₃ or CH₂CH₃; R⁵ is H, Br, Cl, or F; R⁶ and R⁷ are independently
(a) H,
(b) C₁-C₆ alkyl,
(c) hydroxylated C₁-C₆ alkyl,
(d) C₁-C₆ alkoxy-substituted C₁-C₆ alkyl,
(e) halogenated C₁-C₆ alkyl,
(f) aryl, in which aryl represents phenyl, pyridyl, imidazolyl, indolyl, or naphthyl, optionally substituted by one or more substituents selected from halogen, C₁-C₆ alkyl, C₁-C₆ alkoxy, CF₃, OH, C₁-C₆ alkyl-NH—, (C₁-C₆ alkyl)₂-N—, or CN,
(g) aryl substituted C₁-C₆ alkyl, in which aryl represents phenyl, pyridyl, imidazolyl, indolyl, or naphthyl, optionally substituted with one or more substituents selected from halogen, C₁-C₆ alkyl, C₁-C₆ alkoxy, CF₃, or OH,
(h) R⁸—(C₁-C₆)alkyl-, wherein R⁸ is NH₂C═O—, C₁-C₆ alkyl-NHC═O—, (C₁-C₆ alkyl)₂NC═O—, C₁-C₆ alkyl-OOC—, cyano, C₁-C₆ alkyl-CO—NH—, C₁-C₆ alkyl-OOCNH—, C₁-C₆ alkyl-O—, C₇-C₁₂ alkyl-O—C₁-C₆ alkyl-SO—, C₁-C₆ alkyl-S—, C₁-C₆ alkyl-C═O—,—ArCONH—, Ar(C₁-C₆ alkyl)CONH, ArC═O—, NH₂CONH—C₁-C₆ alkyl-NHCONH—, (C₁-C₆ alkyl)₂-NCONH—, ArNHCONH—, hydroxylated C₁-C₆ alkyl-O— or morpholinyl; wherein Ar represents phenyl, pyridyl, imidazolyl, indolyl, or naphthyl optionally substituted with one or more substituents selected from halogen, C₁-C₆ alkyl, C₁-C₆ alkoxy, CF₃, OH, CN,
(i) C₇-C₁₂ alkyl,
OH,
(k) R¹¹—(C₁-C₆) alkyl-COO—(C₁-C₆) alkyl- wherein R¹¹ is HOOC—, or C₁-C₆ alkyl-OOC.

In a more preferred embodiment of the present invention, R¹ is
(a) H,
(b) CH₃, or
(c) CH₂OH;

### R² is

(a) CH₃
(b) CH₂CH₃

### R³ is

(a) H
(b) C₁-C₆ alkyl,
(c) hydroxylated C₁-C₆ alkyl
(d) halogen

### R⁴ is

(a) H,
(b) C₁-C₆ alkyl,
(c) hydroxylated C₁-C₆ alkyl, or
(d) halogen;

### R⁵ is

(a) H, or
(b) halogen;
   R⁶, R⁷ are the same or different
(a) H,
(b) C₁-C₆ alkyl;
(c) hydroxylated C₁-C₆ alkyl
(d) C₁-C₆ alkoxy-substituted C₁-C₆ alkyl

### X is

(a) NH, or
(b) O.

In a more preferred embodiment of the present invention, R¹ and R² are CH₃, R³ and R⁴ are the same or different C₁-C₆ alkyl, R⁵ is hydrogen, R⁶ and R⁷ are the same or different H, C₁-C₆ alkyl, hydroxylated C₁-C₆ alkyl, C₁-C₆ alkoxy-substituted or C₁-C₆ alkyl; and X is NH, or O.

As used herein, the term “C₁-C₆ alkyl” denotes a straight or branched alkyl group having from 1 to 6 carbon atoms. Examples of said C₁-C₆ alkyl include methyl, ethyl, n-propyl, iso-propyl, n-butyl, iso-butyl, sec-butyl, t-butyl and straight- and branched-chain pentyl and hexyl.

The term “halogen” includes fluoro, chloro, bromo and iodo.

The term “medicament induced gastric ulcer” consists of gastric ulcer induced or associated with the use of a medicament e.g. a medicant chosen from a group consisting of NSAID, COX-2 inhibitor, NO-NSAID, and bisphosphonates.

The term “prevent” or “prevention” is given its ordinary meaning and thus means the avoidance or alleviation of the serious consequences of a disease or a side-effect by early detection.

The pure enantiomers, racemic mixtures and unequal mixtures of two enantiomers are within the scope of the invention. It should be understood that all the diastereomeric forms possible (pure enantiomers, racemic mixtures and unequal mixtures of two enantiomers) are within the scope of the invention.

6-carboxamido-imidazo[1,2-a]pyridine of formula I above can thus be used in combination with NSAIDs and deliver the pharmaceutical effect of NSAID and surprisingly avoid the inherent noxious effect NSAIDS have on the stomach linen. It should be appreciated that there is no requirement that the components of the combination according to the present invention must be dosed simultaneously. Sequential or separate use of the components may also provide the desired beneficial effect. Where the administration is sequential, or separate, the delay in administering the second component should not be such as to lose the benefit of the synergistic effect of the combination. 6-carboxamido-imidazo[1,2-a]pyridine compounds of formula I can thus be administered simultaneously, sequentially or separately with an NSAID in therapy, e.g. for the treatment or prophylaxis of arthritis.

COX-2 inhibitors, the newest class of NSAIDS, work by blocking COX-2 enzyme which is involved in the inflammation pathway. By sparing COX-1 enzyme, gastrointestinal toxicity is reduced. A further aspect of the present invention is the combination of the 6-carboxamido-imidazo[1,2-a]pyridine compounds of formula I with COX-2 inhibitors in therapy e.g. for the treatment or prophylaxis of arthritis. Sequential or separate use of the components may also provide the desired beneficial effect. Where the administration is sequential, or separate, the delay in administering the second component should not be such as to lose the benefit of the synergistic effect of the combination. 6-carboxamido-imidazo[1,2-a]pyridine compounds of formula I can thus be administered simultaneously, sequentially or separately with a COX-2 inhibitor for the treatment or prophylaxis of e.g. arthritis.

Nitric oxide releasing NSAIDs (NO-NSAIDs) are disclosed in e.g. WO 94/04484. A further aspect of the present invention is the combination of the 6-carboxamido-imidazo[1,2-a]pyridine compounds of formula I with an NO-NSAID e.g. for the treatment or prophylaxis of pain. Sequential or separate use of the components may also provide the desired beneficial effect. Where the administration is sequential, or separate, the delay in administering the second component should not be such as to lose the benefit of the synergistic effect of the combination. 6-carboxamido-imidazo[1,2-a]pyridine compounds of formula I can thus be administered simultaneously, sequentially or separately with an NO—NSAID for the treatment or prophylaxis of pain.

A further aspect of the present invention is the combination of the 6-carboxamido-imidazo[1,2-a]pyridine compounds of formula I with bisphosphonates in therapy e.g. for the treatment or prophylaxis of osteoporosis. Sequential or separate use of the components may also provide the desired beneficial effect. Where the administration is sequential, or separate, the delay in administering the second component should not be such as to lose the benefit of the synergistic effect of the combination-6-carboxamido-imidazo[1,2-a]pyridine compounds of formula I can thus be administered simultaneously, sequentially or separately with a bisphosphonate compound for the treatment or prophylaxis of e.g. osteoporosis.

Another object of the present invention is the use of certain 1,2,3,4-tetra-hydroisoquinolin-2-yl)pyrimidine compounds of formula II

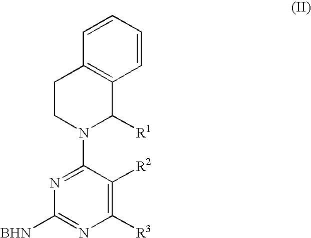

wherein R¹, R² and R³ are independently selected from hydrogen or C₁-C₃ alkyl; and
B is C₁-C₃ alkyl, C₂-C₄ alkenyl, C₃-C₇ cycloalkyl, C₁-C₃ alkoxyethyl, substituted or un-substituted phenylethyl, 3-trifluoromethylphenylmethyl, 4-fluorophenyl, 1-naphthylmethyl, 4-methylthiazol-2-yl or 4-phenylthiazol-2-yl;
in the prevention of medicament induced gastric ulcer.

In a more preferred embodiment of the present invention, R¹, R² and R³ of formula II are all methyl and B is 4-fluorophenyl.

Another object of the present invention is the use of certain tricyclic imidazo[1,2-a]pyridine compounds of formula III

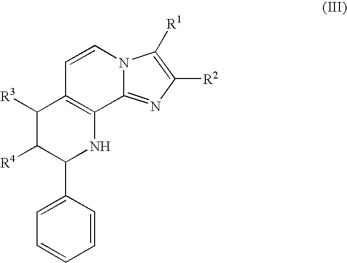

wherein
R¹ is hydroxy C₁-C₄ alkyl;
R² is C₁-C₄ alkyl;
R³ and R⁴ are independently selected from hydrogen, hydroxy, C₁-C₄ alkoxy, halogenated C₁-C₄ alkoxy, C₁-C₄ alkoxy-C₁-C₄ alkoxy, halogenated C₁-C₄ alkoxy-C₁-C₄ alkoxy, C₁-C₄ alkylcarbonyloxy, halogenated C₁-C₄ alkylcarbonyloxy, or carbonyl; in the prevention of medicament induced gastric ulcer.

In a more preferred embodiment of the present invention R¹ is hydroxymethyl; R² is methyl; R³ and R⁴ are independently selected from hydrogen, hydroxy, C₁-C₄ alkoxy or C₁-C₄ alkoxy-C₁-C₄ alkoxy.

Another object of the present invention is the use of certain pyrrolopyridazine compounds of formula IV

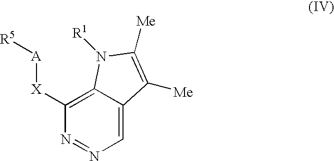

wherein
R¹ is 1-propenyl, 2-propenyl, 1-butenyl, 2-butenyl, 2-methyl-2-propenyl, 3-phenyl-2-propenyl, cyclo-propylmethyl, or 2-methylcyclopropylmethyl;
R⁵ is a phenyl group optionally substituted with halogen;
A is methylene; and
X is oxygen;
in the prevention of medicament induced gastric ulcer.

A more preferred embodiment of the present invention is the use of certain pyrrolopyridazine compounds of formula IV, wherein R¹ is 2-methylcyclopropylmethyl, and R⁵ is a p-fluorophenyl, A is methylene; and X is oxygen.

Another object of the present invention is the use of the 6-carboxamido-imidazo[1,2-a]pyridine compounds of Formula I, as well as pharmaceutically acceptable salts thereof, for the manufacture of a medicament for the prevention of NSAID induced gastric ulcer.

Another object of the present invention is the use of a compound chosen from the group consisting of 6-carboxamido-imidazo[1,2-a]pyridine compounds of Formula I 1,2,3,4-tetra-hydroisoquinolin-2-yl)pyrimidine compounds of formula II, tricyclic imidazo[1,2-a]pyridine compounds of formula III, and pyrrolopyridazine compounds of formula IV for the manufacture of a medicament for the prevention of medicament induced gastric ulcer.

Another object of the present invention is the simultaneous, separate or sequential co-administration of NSAID with the 6-carboxamido-imidazo[1,2-a]pyridine compounds of Formula I for the prevention of NSAID induced gastric ulcer.

Another object of the present invention is the simultaneous, separate or sequential co-administration of a medicament chosen from the group consisting of NSAID, COX-2 inhibitor, NO-NSAID or bisphosphonate with a compound chosen from the group consisting of 6-carboxamido-imidazo[1,2-a]pyridine compounds of Formula I 1,2,3,4-tetra-hydroisoquinolin-2-yl)pyrimidine compounds of formula II, tricyclic imidazo[1,2-a]pyridine compounds of formula III, and pyrrolopyridazine compounds of formula IV for the prevention of medicament induced gastric ulcer.

Still a further object of the present invention is a method for the prevention of NSAID induced gastric ulcer, whereby an effective amount of the 6-carboxamido-imidazo[1,2-a]pyridine compounds of Formula I, as well as pharmaceutically acceptable salts thereof, as active agent is administered simultaneous, separate or sequential with an NSAID to a mammal.

Still a further object of the present invention is a method for the prevention of medicament induced gastric ulcer, whereby an effective amount of a compound chosen from the group consisting of 6-carboxamido-imidazo[1,2-a]pyridine compounds of Formula I 1,2,3,4-tetra-hydroisoquinolin-2-yl)pyrimidine compounds of formula II, tricyclic imidazo[1,2-a]pyridine compounds of formula III, and pyrrolopyridazine compounds of formula IV as active agent is administered simultaneous, separate or sequential with a medicament chosen from a group consisting of COX-2 inhibitor, NO-NSAID, and bisphosphonate to a mammal.

The present invention also relates to an oral pharmaceutical composition for simultaneous administration comprising an NSAID together with a 6-carboxamido-imidazo[1,2-a]pyridine compound of Formula I to prevent NSAID induced gastric ulcer in a mammal.

The present invention also relates to an oral pharmaceutical composition for simultaneous administration comprising a medicament chosen from a group consisting of NSAID, COX-2 inhibitor, NO-NSAID, and bisphosphonate together with a compound chosen from the group consisting of 6-carboxamido-imidazo[1,2-a]pyridine compounds of Formula I, 1,2,3,4-tetra-hydroisoquinolin-2-yl)pyrimidine compounds of formula II, tricyclic imidazo[1,2-a]pyridine compounds of formula III, and pyrrolopyridazine compounds of formula IV to prevent medicament induced gastric ulcer in a mammal.

A pharmaceutical formulation comprising an medicament chosen from a group consisting of NSAID, COX-2 inhibitor, NO-NSAID, and bisphosphonate together with a compound chosen from the group consisting of 6-carboxamido-imidazo[1,2-a]pyridine compounds of Formula I 1,2,3,4-tetra-hydroisoquinolin-2-yl)pyrimidine compounds of formula II, tricyclic imidazo[1,2-a]pyridine compounds of formula III, and pyrrolopyridazine compounds of formula IV as the pharmaceutical active ingredients, may contain further pharmaceutically acceptable carriers, diluents or adjuvants. The pharmaceutical formulation is preferable administered orally.

The amount of the pharmaceutical active ingredients in the pharmaceutical formulation to prevent medicament induced gastric ulcer is an amount which varies according to the mammal being treated, the severity of the disease, the included pharmaceutical active ingredients, and the route of administration selected. Usually the amount of pharmaceutical active ingredients are between 0.1-95% by weight of the preparation, preferably between 0.1-20% by weight in preparations for parenteral use and preferably between 0.1 and 50% by weight in preparations for oral administration.

The present invention also relates to an oral pharmaceutical composition for simultaneous administration comprising a COX-2 inhibitor together with a 6-carboxamido-imidazo[1,2-a]pyridine compound of Formula I in therapy, e.g. to prevent induced gastric ulcer in a mammal.

A pharmaceutical formulation comprising a COX-2 inhibitor together with the 6-carboxamido-imidazo[1,2-a]pyridine compound of Formula I as the pharmaceutical active ingredients, may contain further pharmaceutically acceptable carriers, diluents or adjuvants. The pharmaceutical formulation is preferable administered orally.

The present invention also relates to an oral pharmaceutical composition for simultaneous administration comprising an NO-NSAID together with a 6-carboxamido-imidazo[1,2-a]pyridine compound of Formula I in therapy, e.g. to prevent, induced gastric ulcer in a mammal.

A pharmaceutical formulation comprising a an NO-NSAID together with the 6-carboxamido-imidazo[1,2-a]pyridine compound of Formula I as the pharmaceutical active ingredients, may contain further pharmaceutically acceptable carriers, diluents or adjuvants. The pharmaceutical formulation is preferable administered orally.

The present invention also relates to a kit comprising a dosage unit of a compound chosen from the group consisting of 6-carboxamido-imidazo[1,2-a]pyridine compounds of Formula I 1,2,3,4-tetra-hydroisoquinolin-2-yl)pyrimidine compounds of formula II, tricyclic imidazo[1,2-a]pyridine compounds of formula II, and pyrrolopyridazine compounds of formula IV and a dosage unit of a an NSAID, a COX-2 inhibitor, an NO-NSAID, or an bisphosphonate optionally with instructions for use.

Examples of NSAID to be used in the present invention include, but is not limited to,

Diclofenac,
Diflunisal,
Etodolac,
Fenoprofen,
Floctafenine,
Flurbiprofen,
Ibuprofen,
Indomethacin,
Ketoprofen,
Meclofenamate,
Mefenamic Acid,
Meloxicam,
Nabumetone,
Naproxen,
Oxaprozin,
Phenylbutazone,
Piroxicam,
Sulindac,
Tenoxicam,
Tiaprofenic Acid, and
Tolmetin

Examples of COX-2 inhibitors to be used in the present invention include, but is not limited to, Celebrex (Celecoxib), Vioxx (Rofecoxib).

Examples of NO-NSAID to be used in the present invention include, but is not limited to, those disclosed in WO 96/32946, WO 96/35416, WO 96/38136, WO 96/39409, WO00150037, U.S. Pat. No. 6,057,347, WO 94/04484, WO 94/12463, WO 95/09831, WO 95/30641, WO 97/31654, WO 99/44595 and WO 99/45004.

Examples of bisphosphonates to be used in the present invention include, but are not limited to, alendronate, risedronate, tiludronate, ibandronate, zoledronate and etidronate.

The invention is illustrated, but in no way limited, by the following examples.

### EXAMPLES

Groups of 10 male rats were given oral doses of vehicle, 2,3-dimethyl-8-(2-ethyl-6-methylbenzylamino)-imidazo[1,2-a]pyridine-6-carboxamide(0.3, 1, 3 and 10 μmol/kg) or ranitidine (10 μmol/kg). Indomethacin 20 mg/kg, orally) was given 1 h after dosing. Stomach was removed 5 h after indomethacin and examined macroscopically

### RESULTS

Indomethacin induced ulcers in the corpus only, rumen and anthrum were unaffected. Ulcers in the corpus were classified as pinhead (diameter 3 mm or less) or furrows (>3 mm).

2,3-dimethyl-8-(2-ethyl-6-methylbenzylamino)-imidazo[1,2-a]pyridine-6-carboxamide had a protective effect against gastric ulcers induced by indomethacin. This protective effect was dose-dependent and characterised by a decrease in the number of pinhead ulcers and ulcer furrows in the corpus. The decrease was statistically significant from the dose of 3 μmol/kg and maximal at 10 μmol/kg. Ranitidine had no effect.

Median number of gastric (corpus)
ulcers induced by indomethacin

| | Pinhead | Ulcer |
|---|---|---|
| Group | ulcers | furrows |
| Vehicle | 5 | 9 |
| 2,3-dimethyl-8-(2-ethyl-6-methylbenzylamino)- | 5 | 8 |
| imidazo[1,2-a]pyridine-6-carboxamide | | |
| [0.3 μmol/kg] | | |
| 2,3-dimethyl-8-(2-ethyl-6-methylbenzylamino)- | 5 | 9 |
| imidazo[1,2-a]pyridine-6-carboxamide | | |
| [1 μmol/kg] | | |
| 2,3-dimethyl-8-(2-ethyl-6-methylbenzylamino)- | 2 | 1 |
| imidazo[1,2-a]pyridine-6-carboxamide | | |
| [3 μmol/kg] | | |
| 2,3-dimethyl-8-(2-ethyl-6-methylbenzylamino)- | 0 | 0 |
| imidazo[1,2-a]pyridine-6-carboxamide | | |
| [10 μmol/kg] | | |
| Ranitidine | 7 | 11 |
| [10 μmol/kg] | | |

1. Use of a compound of formula I 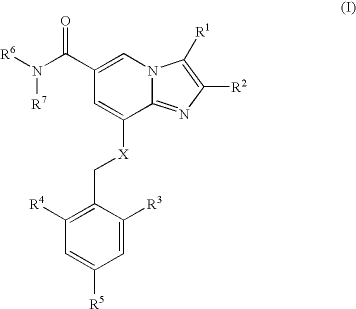
or a pharmaceutically acceptable salt thereof, wherein
R¹ is
(a) H,
(b)CH₃, or
(c)CH₂OH;
R² is
(a) CH₃ or
(b) CH₂CH₃;
R³ is
(a) H,
(b) C₁-C₆ alkyl,
(c) hydroxylated C₁-C₆ alkyl, or
(d) halogen;
R⁴ is
(a) H,
(b) C₁-C₆ alkyl,
(c) hydroxylated C₁-C₆ alkyl, or
(d) halogen;
R⁵ is
(a) H, or
(b) halogen;
R⁶ and R⁷ are independently selected substituents, containing C, H, N, O, S, Se, P and halogen atoms, which give compounds of Formula I a molecular weight≦600,
X is
(a) NH, or
(b) O,
in the prevention of medicament induced gastric ulcer.

2. Use according to claim 1 wherein
R¹ is CH₃ or CH₂OH;
R² is CH₃,
R³ is CH₃ or CH₂CH₃;
R⁴ is CH₃ or CH₂CH₃;
R⁵ is H, Br, Cl, or F;
R⁶ and R⁷ are independently
(a) H,
(b) C₁-C₆ alkyl,
(c) hydroxylated C₁-C₆ alkyl,
(d) C₁-C₆ alkoxy-substituted C₁-C₆ alkyl,
(e) halogenated C₁-C₆ alkyl,
(f) aryl, in which aryl represents phenyl, pyridyl, imidazolyl, indolyl, or naphthyl, optionally substituted by one or more substituents selected from halogen, C₁-C₆ alkyl, C₁-C₆ alkoxy, CF₃, OH, C₁-C₆ alkyl-NH—, (C₁-C₆ alkyl)₂-N—, or CN—,
(g) aryl substituted C₁-C₆ alkyl, in which aryl represents phenyl, pyridyl, imidazolyl, indolyl, or naphthyl, optionally substituted with one or more substituents selected from halogen, C₁-C₆ alkyl, C₁-C₆ alkoxy, CF₃, or OH,
(h) R⁸—(C₁-C₆) alkyl-, wherein R⁸ is NH₂C═O—, C₁-C₆ alkyl-NHC═O—, (C₁-C₆ alkyl)₂NC═O—, C₁ C₆ alkyl OOC, cyano, C₁ C₆ alkyl-CO—NH—, C₁-C₆ alkyl-OOCNH—, C₁-C₆ alkyl-O—, C₇-C₁₂ alkyl-O— C₁-C₆ alkyl-SO—, C₁-C₆ alkyl-S—, C₁-C₆ alkyl-C═O—, ArCONH—, Ar(C₁-C₆ alkyl)CONH, ArC═O—, NH₂CONH—C₁-C₆ alkyl-NIICONH—, (C₁-C₆ alkyl)₂-CONH—, ArNHCONH—, hiydroxylated C₁-C₆ alkyl-O—, or morpholinyl; wherein Ar represents phenyl, pyridyl, imidazolyl, indolyl, or naphthyl optionally substituted with one or more substituents selected from halogen, C₁-C₆ alkyl, C₁-C₆ alkoxy, CF₃, OH, CN,
(i) C₇-C₁₂ alkyl,
(j) OH,
(k) R¹¹—(C₁-C₆) alkyl-COO—(C₁-C₆) alkyl- wherein R¹¹ is HOOC—, or C₁-C₆ alkyl-OOC.

3. Use according to claim 1 wherein
R¹ is
(a) H,
(b) CH₃, or
(c)CH₂OH;
R² is
(a)CH₃
(b) CH₂CH₃
R³ is
(a) H,
(b) C₁-C₆ alkyl,
(c) hydroxylated C₁-C₆ alkyl
(d) halogen
R⁴ is
(a) H, or
(b) C₁-C₆ alkyl,
(c) hydroxylated C₁-C₆ alkyl, or
(d) halogen;
R⁵ is
(a) H, or
(b) halogen;
R⁶, R⁷ are the same or different
(a) H,
(b) C₁-C₆ alkyl;
(c) hydroxylated C₁-C₆ alkyl
(d) C₁-C₆ alkoxy-substituted C₁-C₆ alkyl
X is
(a) NH, or
(b) O.

4. Use according to claim 1, wherein
R¹ and R² are CH₃,
R³ and R⁴ are the same or different C₁-C₆ alkyl,
R⁵ is hydrogen,
R⁶ and R⁷ are the same or different H, C₁-C₆ alkyl, hydroxylated C₁-C₆ alkyl, C₁-C₆ alkoxy-substituted or C₁-C₆ alkyl; and
X is NH, or O.

5. Use of a compound of formula II, 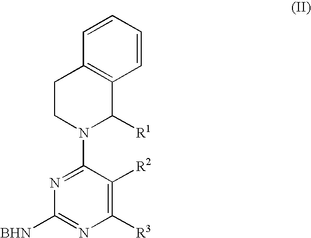
or a pharmaceutically acceptable salt thereof, wherein
R¹, R² and R³ are independently selected from hydrogen or C₁-C₃ alkyl; and
B is C₁-C₃ alkyl, C₂-C₄ alkenyl, C₃-C₇ cycloalkyl, C₁-C₃ alkoxyethyl, substituted or unsubstituted phenylethyl, 3-trifluoromethylphenylmethyl, 4-fluorophenyl, 1-naphthylmethyl, 4-methylthiazol-2-yl or 4-phenylthiazol-2-yl; in the prevention of medicament induced gastric ulcer.

6. Use according to claim 5, wherein R¹, R² and R³ are all methyl and B is 4-fluorophenyl.

7. Use of a compound of formula III, 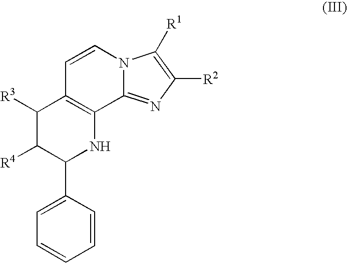
wherein
R¹ is hydroxy C₁-C₄ alkyl;
R² is C₁-C₄ alkyl;
R³ aid R⁴ are independently selected from hydrogen, hydroxy, C₁-C₄ alkoxy, halogenated C₁-C₄ alkoxy, C₁-C₄ alkoxy-C₁-C₄ alkoxy, halogenated C₁-C₄ alkoxy-C₁-C₄ alkoxy, C₁-C₄ alkylcarbonyloxy, halogenated C₁-C₄ alkylcarbonyloxy, or carbonyl; in the prevention of medicament induced gastric ulcer.

8. Use according to claim 7, wherein R¹ is hydroxymethyl; R² is methyl; R³ and R⁴ are independently selected from hydrogen, hydroxy, C₁-C₄ alkoxy or C₁-C₄ alkoxy-C₁-C₄ alkoxy.

9. Use of a compound of formula IV, 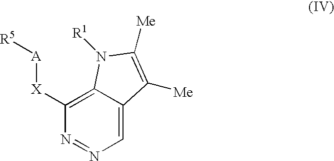
wherein
R¹ is 1-propenyl, 2-propenyl, 1-butenyl, 2-butenyl, 2-methyl-2-propenyl, 3-phenyl-2-propenyl, cyclo-propylmethyl, or 2-methyl-cyclopropylmethyl;
R⁵ is a phenyl group optionally substituted with halogen;
A is methylene; and
X is oxygen; in the prevention of medicament induced gastric ulcer.

10. Use according to claim 9, wherein
R¹ is 2-methylcyclopropylmethyl, and
R⁵ is a p-fluorophenyl,
A is methylene; and X
is oxygen.

11. A combination comprising a compound as defined in any one of claims 1, 5, 7 and 9; and an NSAID for simultaneous, sequential or separate use in therapy.

12. A combination comprising a compound as defined in any one of claims 1, 5, 7 and 9; and a COX-2 inhibitor for simultaneous, sequential or separate use in therapy.

13. A combination comprising a compound as defined in any one of claims 1, 5, 7 and 9; and an NO-NSAID for simultaneous, sequential or separate use in therapy.

14. A combination comprising a compound as defined in any one of claims 1, 5, 7 and 9; and a bisphosphonate for simultaneous, sequential or separate use in therapy.

15. A pharmaceutical formulation comprising the combination according to claim 11 and a pharmaceutically acceptable carrier or diluent.

16. A first pharmaceutical formulation comprising a compound as defined in any one of claims 1, 5, 7 and 9 and a pharmaceutically acceptable carrier or diluent; and a second pharmaceutical formulation comprising an NSAID, a COX-2 inhibitor, a bisphosphonate or an NO-NSAID and a pharmaceutically acceptable carrier or diluent.

17. A kit comprising a dosage unit of a compound as defined in any one of claims 1, 5, 7 and 9; and a dosage unit of an NSAID, a COX-2 inhibitor, an NO-NSAID or a bisphosphonate, optionally with instructions for use.

18. (canceled)

19. Method for prevention of medicament induced gastric ulcer, whereby a compound according to any one of claims 1, 5, 7 and 9 as active agent is administered simultaneous, separate or sequential with an NSAID, a COX-2 inhibitor, an NO-NSAID or a bisphosphonate to a mammal.

20. An oral pharmaceutical composition in unit dosage form for the prevention of medicament induced gastric ulcer in a mammal comprising either an NSAID, a COX-2 inhibitor, an NO-NSAID or a bisphosphonate together with a compound of any one of claims 1, 5, 7 and 9.
